(19) United States
(12) Plant Patent Application Publication
(10) Pub. No.: US 2014/0090131 P1
(43) Pub. Date: Mar. 27, 2014

(54) Phalaenopsis orchid plant named 'Ava Star'

(50) ;

(71) Applicant: Floricultura, Heemskerk (NL)

(72) Inventor: René Schoone, Assendelft (NL)

(73) Assignee: Floricultura, Heemskerk (NL)

(21) Appl. No.: 13/998,016

(22) Filed: Sep. 24, 2013

Related U.S. Application Data
(60) Provisional application No. 61/705,229, filed on Sep. 25, 2012.

(30) Foreign Application Priority Data
Sep. 27, 2012 (NL) OPS971

(51) Int. Cl. A01H 5/00

(52) U.S. Cl. PLT/311

(57) ABSTRACT

A new and distinct Phalaenopsis plant named ‘Ava Star’ particularly characterized by flowers which are white with a red/purple labellum; plants which may be propagated economically and uniformly using tissue culture; plants which produce more than one inflorescence; long and sturdy inflorescences; and relatively short, dark-green foliage.

### LATIN NAME OF THE GENUS AND SPECIES OF THE PLANT CLAIMED

Phalaenopsis hybrid.

### VARIETY DENOMINATION

‘Ava Star’

### BACKGROUND OF THE INVENTION

The present invention relates to a new and distinct cultivar of Phalaenopsis plant, botanically known as Phalaenopsis of the Orchidaceae family, and hereinafter referred to by the cultivar name ‘Ava Star’.

Phalaenopsis comprises a genus of about 55 species of herbaceous perennials many of which, or the hybrids thereof, are suitable for cultivation in the home or greenhouse. Phalaenopsis is predominantly epiphytic or rock-dwelling, and is native to tropical Asia, the Malay Archipelago, and Oceania. The species typically has 2-ranked, fleshy, oblong or elliptic leaves affixed to a short central stem (monopodial growth), which vary in size from 5 to 8 inches to over 2 feet. The leaves may be entirely green or mottled with silver grey.

Phalaenopsis orchids, often referred to as ‘Moth Orchids’ in the horticultural trade, are frequently used to furnish cut flowers for the florist trade or sold as flowering potted-plants for home or interiorscape.

Phalaenopsis produces upright or pendent lateral racemes, often with many showy flowers which open in succession beginning with the lowermost. The flowers possess three sepals and three petals; the lateral ones being alike. The lowermost petal, called the labellum, is three-lobed and is often more brightly-colored than the other flower segments. Flower colors include various shades of pink, white, yellow and red-brown.

Phalaenopsis orchids are typically propagated from seeds. Asexual propagation of Phalaenopsis is often done from off-shoots which frequently arise from the lower bracts of the inflorescence. The resulting plants are detached from the mother plant and may be planted in a suitable substrate.

The new Phalaenopsis ‘Ava Star’ is a product of a controlled breeding program conducted by the inventor, René Schoone, in Strengweg, Heemskerk, The Netherlands. The objective of the breeding program was to develop a new Phalaenopsis cultivar particularly characterized by its attractive and unique colored flowers, economical propagation via tissue culture, rapid growth, and a plant dimension suitable for packaging and shipping to the market.

The new Phalaenopsis ‘Ava Star’ originated from a cross made by the inventor in 2000 in Strengweg, Heemskerk, The Netherlands. The female or seed parent is the Phalaenopsis cultivar designated ‘Percy Porter’, unpatented. The male or pollen parent is the Phalaenopsis cultivar designated ‘Red Eye’, unpatented. The new Phalaenopsis ‘Ava Star’ was discovered and selected by the inventor as a single flowering plant within the progeny of the stated cross in a controlled environment in 2008 in Strengweg, Heemskerk, The Netherlands.

Asexual reproduction of the new Phalaenopsis cultivar by tissue culture was first performed in July, 2008 in Cieweg 13, Heemskerk, The Netherlands, and has demonstrated that the combination of characteristics as herein disclosed for the new cultivar are firmly fixed and retained through successive generations of asexual reproduction. The new cultivar asexually reproduces true-to-type.

### BRIEF DESCRIPTION OF THE INVENTION

The following traits have been repeatedly observed and are determined to be unique characteristics of ‘Ava Star’, which in combination distinguish this Phalaenopsis as a new and distinct cultivar:

1. flowers which are white with a red/purple labellum;

2. plant produces more than one inflorescence;

3. plants may be propagated economically and uniformly using tissue culture;

4. inflorescences are long and sturdy; and

5. relatively short, dark-green foliage.

In comparison with the parental cultivars of ‘Ava Star’, the female parent ‘Percy Porter’ has white flowers and they are about 12 cm in size. The male parent ‘Red Eye’ has white colored flowers with a purple labellum and they are about 9 cm in size, whereas the flowers of ‘Ava Star’ are white with a red/purple labellum and are about 10.5 cm in size.

Presently, there is no commercial cultivar to which ‘Ava Star’ can be meaningfully compared.

### BRIEF DESCRIPTION OF THE PHOTOGRAPHS

The accompanying photographs illustrate the overall appearance of the new Phalaenopsis ‘Ava Star’ showing the colors as true as is reasonably possible with colored reproductions of this type. Colors in the photographs may differ slightly from the color values cited in the detailed botanical description, which accurately describe the color of ‘Ava Star’.

FIG. 1 shows a side view perspective of a typical flowering plant of ‘Ava Star’ in a 12 cm pot, at 16 months of age.

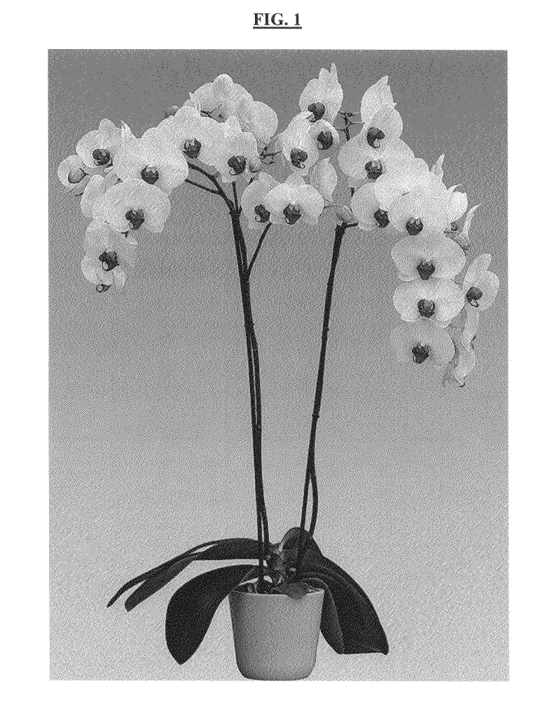

FIG. 2 shows a close-up view of the typical buds and flowers of ‘Ava Star’.

FIG. 3 shows a close-up view of the typical leaves of ‘Ava Star’.

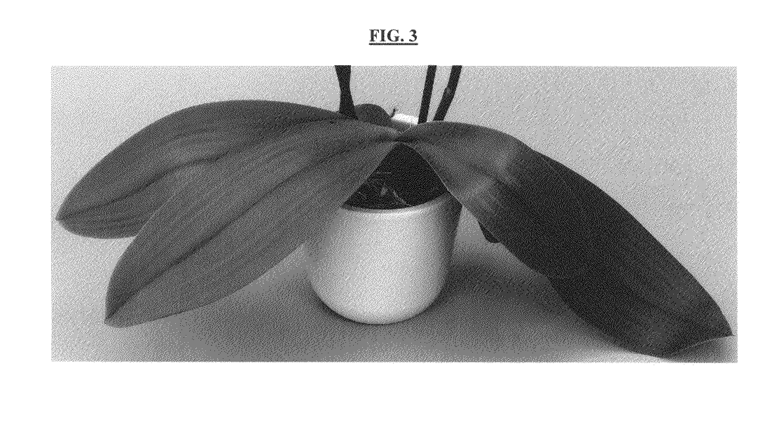

### DETAILED BOTANICAL DESCRIPTION

The new Phalaenopsis cultivar ‘Ava Star’ has not been observed under all possible environmental conditions. The phenotype of the new cultivar may vary with variations in environment such as temperature, light intensity, and day length without any change in the genotype of the plant.

The aforementioned photographs, together with the following observations, measurements and values describe plants of ‘Ava Star’ as grown in a greenhouse in Strengweg, Heemskerk, The Netherlands, under conditions which closely approximate those generally used in commercial practice. Initially, the ideal temperature to grow plants of ‘Ava Star’ is 27° C. during the day and at night. Then, during the flowering phase of ‘Ava Star’, the ideal growing temperature is 20-22° C. during the day and 18° C. at night. Light levels for growing ‘Ava Star’ are a minimum of 5,000 lux and a maximum of 10,000 lux. A balanced fertilizer with level of 200 ppm N, 87 ppm P, 168 ppm K is applied. Duration of growth of ‘Ava Star’ from potting size is between 10 and 14 months.

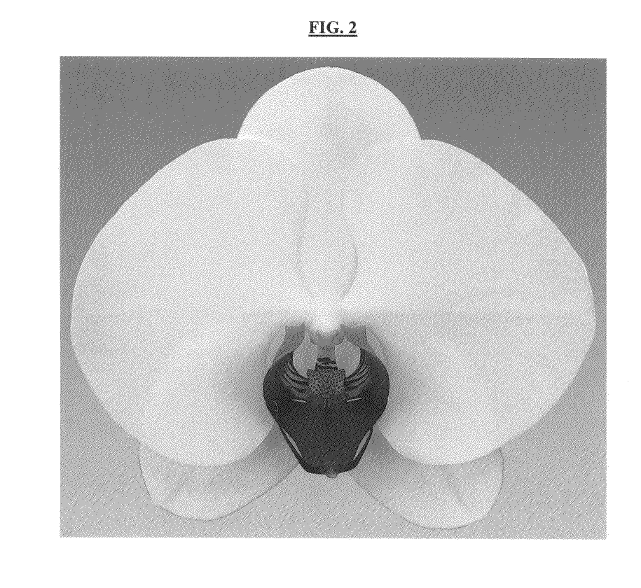

Color references are made to The Royal Horticultural Society Colour Chart (R.H.S.), 2007 edition, except where general colors of ordinary significance are used. Color values were taken under daylight conditions at approximately noon in Zaandammerweg, Assendelft, The Netherlands. The age of the ‘Ava Star’ plants described is 12 months after potting.
Classification:
   Botanical.—Phalaenopsis hybrid.
Parentage:
   Female or seed parent.—Phalaenopsis cultivar designated ‘Percy Porter’, unpatented.
   Male or pollen parent.—Phalaenopsis cultivar designated ‘Red Eye’, unpatented.
Propagation:
   Type.—tissue culture.
   Rooting habit and description.—approximately 4 mm-6 mm wide and grey/green (RHS 193C) in color; freely branching. It takes 12 weeks for plants growing in tissue culture to initiate roots.
Plant:
   Size at maturity.—Height: about 70 cm to 90 cm Spread: about 50 to 70 cm.
   Growth habit.—large; green (RHS 143B) leaves and a relatively normal raceme.
   Vigor.—moderate.
   Crop time.—Following asexual propagation, at about 26 weeks 2 leaves appear; at about 30 weeks 3-4 leaves appear; after a cold treatment of about 4-8 weeks at a temperature of about 19° C. about 2 peduncles with flowers appear.
Foliage:
   Quantity per plant.—About 6 to 8 leaves are produced before flowering.
   Arrangement and attachment.—half up/horizontal and on two sides.
   Overall shape of leaf.—oval, the tip is little pointy and asymmetric.
   Texture.—smooth and leathery.
   Pubescence.—none.
   Mature leaf length.—about 15 to 30 cm.
   Mature leaf width.—about 6 to 10 cm.
   Mature leaf thickness.—about 2 mm.
   Mature leaf color.—upper side: RHS 141B Under side: RHS 143B.
   Leaf base.—acute.
   Margin.—entire.
   Venation.—Pattern: parallel Color of midvein: upper side: green (RHS 141B) Under side: green (RHS 143B).
Raceme:
   Quantity per plant.—about 1 to 2.
   Number of flowers per raceme.—about 10 to 25.
   Length.—about 85 cm.
   Diameter.—about 6 mm.
   Strength.—strong.
   Aspect.—upright.
   Texture.—glabrous and smooth.
   Color.—combination of RHS 146D & RHS N200A.
   Internode.—Length: about 45 mm.
Inflorescence description:
   Appearance.—upright to slightly pendant, racemose inflorescence with bilaterally symmetrical flowers that open in succession beginning with the lowermost flower.
   Buds.—Height (from base to tip): about 30 mm Diameter (at midpoint): about 24 mm Shape: oval/egg-shaped Color: RHS 154C.
   Flowering time.—For an untreated plant (flowering plant that has not undergone cold-treatment where the plant grows at a temperature of 18° C. to 19° C. for about 4 to 8 weeks after a period of about 30 weeks at a temperature of 25° C.), 2 racemes appear with about 16 to 20 flower buds and flowers per inflorescence. First flowers can be expected approximately 4 to 6 months after planting a plant with a leaf diameter of 3 to 5 cm. Flowers persistent.
   Flowering longevity.—On the plant: about 4 to 6 months; lastingness of cut flowers: has not been observed.
   Fragrance.—no fragrance.
   Flower.—Rate of opening: Flowers fully opened about 2 to 3 days after petal and sepal separation. Orientation at opening: slanted upward and outward. Shape: Typical shape of phalaenopsis orchid, see FIG. 2 Size: Height: about 80-100 mm Diameter: about 90-110 mm Depth of tube: about 19 mm.
   Petals.—Quantity and arrangement: six petals that are trimerous, overlapping and arranged in 2 whorls. Petals are more pronounced than sepals Arrangement: Inner whorl of petals comprises 3 petals, 2 lateral petals and labellum. 2 lateral petals: Overall shape: broadly ovate and weakly cupped Apex: round Margin: entire and weakly undulate Base: broadly ovate Length: about 57 mm Width: about 43 mm Texture: Upper surface: smooth and satiny Under surface: smooth and satiny Color (when fully opened): upper and under surface: white (RHS NN155C) Labellum: Overall shape: 3-lobed with 2 prominent callosities at central junction of the lateral lobes and base of the midlobe. Lateral lobes of labellum fold upward about the column; the midlobe extends forward and is terminated by 2 short stubs appendages at the apex. Lateral lobes of the labellum are ovate in shape while the midlobe is triangular with a bump and a rib on it Margin: entire and weakly undulate Apex: oval Length: about 21 mm Width (not flattened): about 25 mm Texture: Upper & under surface: smooth and satiny Color (when fully opened): Midlobe, upper surface: red/purple (RHS 72A). The edge at the base is greyed/red (RHS 179B). Under surface: From top till under in the center white (RHS NN155D), next to that on the sides red/purple (RHS 70B). The outer edge is red/purple (RHS 72A). Upper edge at the base is greyed/purple (RHS 183C) and some yellow/orange (RHS 23C). Lateral lobes, upper surface: At the base white (RHS NN155D) with greyed/purple stripes (RHS 183A) which runs into RHS 71S. At the bottom first RHS 59A, which runs into the main color red/purple (RHS 72B). Under surface: At the base white (RHS NN155D) with purple stripes (RHS N78A). At the bottom red/purple (RHS 59A) which runs into the main color RHS N78C. At the upper edge also a little RHS 23C. Chirri: long with large curl at the end (about 25 mm) color: red/purple (RHS 59A) and tip is white (RHS NN155B) Pestle (Callosities): Length: about 5 mm Width (not flattened): about 7 mm Color: yellow (RHS 13B), with white (RHS NN155C) with red/purple spots (RHS 59A).
   Sepals.—Arrangement: Outer whorl of petals comprises 3 sepals. Overall shape: elliptical and weakly cupped Margin: entire and weakly undulate Length: about 46 mm Width: about 34 mm Apex: oval and little pointy Texture: Upper & under surface: smooth and satiny Color (when fully opened): Dorsal, upper surface: white (RHS NN155C). Under surface: white (RHS NN155D) with a red/purple haze (RHS 72B). Lateral, upper surface: white (RHS NN155C) with a yellow/green haze (RHS 145C. At the base some stripes/spots RHS N78A. Under surface: white (RHS NN155D) with a yellow/green haze (RHS 145C). The midvein is purple (RHS N78A). The edges also have a haze of RHS N78A.
Reproductive organs:
   Arrangement.—The stamens, style and stigmas are fused into a single, short structure called the column, possessing one terminal anther with pollen grains united into a pollinia, which are covered by an anther cap. The stigma is located under the column behind the pollinia. The ovary is inferior with three carpels present. The plant has not produced seed.
   Column.—Length: about 8 mm Diameter: about 5 mm Color: white (RHS NN155B).
   Pollinia.—Quantity: Two Diameter: about 1.5 mm Color: yellow/orange RHS 14B.
   Ovary.—Length: about 4 mm Diameter: about 3 mm Color: RHS NN155C.
   Pedicel.—Length: about 40 mm Diameter: about 3 mm Texture: glabrous and smooth Color: Close to the flower white (RHS NN155C), which runs into yellow/green (RHS 145D).
Pest Resistance/susceptibility: No specific resistance or susceptibility observed
Temperature tolerance: Tolerant to a low temperature of about 15° C. and to a high temperature about 30° C.

What is claimed is:

1. A new and distinct Phalaenopsis plant named ‘Ava Star’, as illustrated and described herein.
